(19) United States
(12) Patent Application Publication
(10) Pub. No.: US 2002/0033362 A1
(43) Pub. Date: Mar. 21, 2002

(54) Apparatus for supplying and sanitizing the water lines of dental units

(75) Inventor: Franco Castellini, Bologna (IT)

Correspondence Address: Richard J. Minnich, Esq., Fay, Sharpe, Fagan, Minnich & McKee, LLP, Seventh Floor, 1100 Superior Avenue, Cleveland, OH, 44114-2518, US

(73) Assignee: Castellini S.p.A.

(21) Appl. No.: 09/955,273

(22) Filed: Sep. 18, 2001

(30) Foreign Application Priority Data
Sep. 19, 2000 (IT) BO2000A 000542

(51) Int. Cl. B01D 21/30

(52) U.S. Cl. 210/97

(57) ABSTRACT

An apparatus for supplying and sanitizing the water line of a dental unit comprises the following: at least one second branch connected and leading into a first branch which supplies a second disinfectant/sterilizing fluid into a main line, the second branch and the main line being equipped with respective second and third shutoff elements designed to supply the first branch with the first fluid and with the second fluid alternately and as required; a first container for holding handpieces during sterilization, disinfection, flushing or cleaning of the water line; and switching means connected to control means that can be operated manually or automatically upon the occurrence of defined events on the dental unit and designed to determine successive manual or automatic operating combinations and/or sequences that may be repeated two or more times during which the first, second and third shutoff elements are opened and closed in such a way as to perform cycles of treatment on the first branch with the first fluid and/or alternatively with the second fluid conveyed by the second branch.

### BACKGROUND OF THE INVENTION

The present invention relates to an apparatus for supplying and sanitizing the water line of a dental unit.

One of the most important features of a dental unit, and one which has undergone considerable development over the years, especially as regards its functionality, is its water system which supplies fluids used by dental equipment and patients (water or physiological saline for tumblers and handpieces), and by consumer units such as the spittoon (water used to clean the spittoon itself).

This development has been mainly prompted by increased standards of hygiene required for this type of equipment. These standards have led to considerable advances being made in the design and function of the water line of a dental unit not only to guarantee their efficient operation and durability but also to maintain the sterility of the conduits both during and between successive patient treatments.

Indeed, at the present time, the water line of a dental unit typically comprises a first main branch for supplying water from a main water source and in turn comprising a plurality of sub-branches leading to the operating and accessory equipment. These branches may be equipped with different systems variously designed to perform different functions and on the basis of different methods all aimed at improving the functioning and disinfection of the water line.

Of these methods and systems, the most common today are those for disinfecting and sterilizing the conduits (for example, a continuous disinfecting/sterilizing cycle is described in patent publications EP-111.249 and EP-317.521, the latter by the Applicant of the present application). A further feature consists in applying to the patient-user supply line a system for continuously draining off the water (see patent EP-368.818) designed to continuously renew the water in the line and preventing it from stagnating inside the line while the dental unit is on.

All these systems have greatly improved the hygienic conditions of the water line of the dental unit, but restricted to the use of a single fluid passing through the water line, and extremely long sanitizing cycles that may in some cases reduce the time in which the dental unit can be used.

The fact that the disinfection and sterilization procedures take a long time to be completed means that the dental unit cannot be quickly restored to working condition or started up in urgent cases when the unit itself is not in ideal conditions in terms of hygiene since the water line may harbor a bacterial burden higher than allowed (due, for example, to fluid that has remained in the system).

The Applicant, thanks to its experience and constant research into the systems and apparatus of the kind described above, has designed a new apparatus for supplying and sanitizing a dental unit and that is extremely simple and economical while at the same time offering a full set of disinfection and sterilization functions that will meet the most diverse requirements of a modern dental surgery without complicating the structure and use of the apparatus.

### SUMMARY OF THE INVENTION

The present invention provides an apparatus for supplying and sanitizing the water line of a dental unit, the apparatus comprising the following: at least one second branch connected and leading into a first branch which feeds a second disinfectant/sterilizing fluid into a main line, the second branch and the main line being equipped with respective second and third shutoff elements designed to supply the first branch with the first fluid or, alternately and as required, with the second fluid; a first container for holding handpieces during sterilization, disinfection, flushing or cleaning of the water line; and switching means connected to control means that can be operated manually or triggered automatically upon the occurrence of defined events on the dental unit and designed to determine successive manual or automatic operating combinations and/or sequences that may be repeated two or more times during which the first, second and third shutoff elements are opened and closed in such a way as to perform cycles of treatment on the first branch with the first fluid and/or alternatively with the second fluid conveyed by the second branch.

### BRIEF DESCRIPTION OF THE DRAWINGS

The technical features of the present invention, in accordance with the above-mentioned aims, are set out in the claims below and the advantages more clearly illustrated in the detailed description which follows, with reference to the accompanying drawings, which illustrate a preferred embodiment of the invention without restricting the scope of the inventive concept, and in which:

FIG. 1 is a diagram of the water line of a dental unit equipped with a supply and sanitizing apparatus made according to the present invention;

FIG. 2 is a scaled-up detail from FIG. 1 in a front view and with some parts cut away in order to better illustrate others.

### DESCRIPTION OF THE PREFERRED EMBODIMENTS

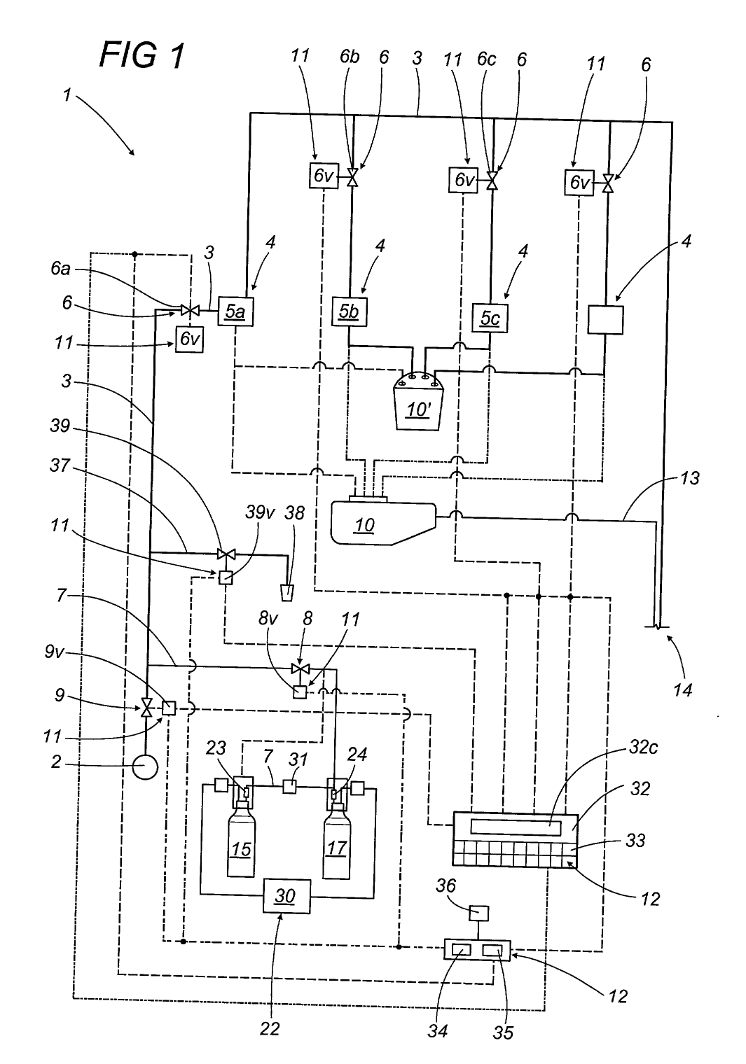

With reference to the accompanying drawings, in particular, FIG. 1, an apparatus for supplying and sanitizing the water line of a dental unit comprises a main fluid supply line, labeled 1 in its entirety and connected at one end to at least one source 2 of a first fluid (usually water from the mains) and at the other end to operating units of the dental unit through at least one first branch 3 for supplying a set of use-points 4 comprising a plurality of handpieces 5.

The handpieces 5 (being of customary type) are illustrated only schematically and may include a syringe 5a, a micromotor 5b, a turbine 5c, etc.

Each of the handpieces 5 is equipped with first elements 6 (customary valves) for shutting off the fluid supply to that handpiece 5 on a control from the dentist. Each of the first shutoff elements (6a, 6b, 6c, etc) is opened when the corresponding handpiece is selected by the dentist.

The water line comprises a second branch 7, a first container 10 and switching means 11 connected to corresponding control means 12.

Looking in more detail (again with reference to the accompanying drawing), the second branch 7 is connected to and leads into the first supply branch 3 so as to allow a second fluid, in particular a disinfectant/sterilizing fluid, to flow through the first branch 3. The second branch 7 and the main line 1 are equipped with respective second and third shutoff elements 8 and 9 (again, customary valves) designed to switch the supply of the first branch 3 between one or other of the two fluids on a control from the dentist working on the dental unit.

The second branch 7, unlike the main line 1, is supplied through a corresponding first tank 15, preferably independent and/or interchangeable, mounted on the dental unit.

The first tank 15 may be connected to the second branch 7, as described in more detail below.

The first tank 15 may be of the disposable type or of the type that can be sterilized and reused.

As shown in FIG. 1, the apparatus disclosed may, to increase its operating capability, comprise a third branch 16 for supplying a third fluid, instead of the first fluid, to the main line 1 and hence to the first connecting branch 3.

The third fluid is preferably contained in a corresponding second tank 17 which, like the first tank 15, is independent and/or interchangeable and of the disposable or sterilizable and reusable type.

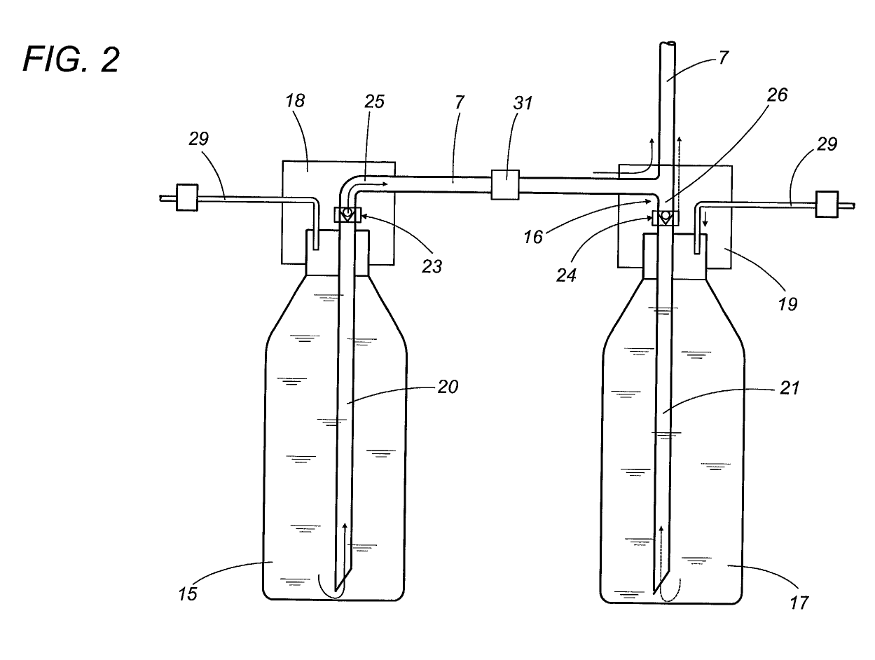

With reference also to FIG. 2, both the second and third branches 7 and 16 are equipped with respective closures 18 and 19 (for example, a kind of cap located in the dental unit) used to connect up the first and second tanks 15 and 17 of the fluids to be supplied.

The tanks 15 and 17 are fitted with tubes 20 and 21, respectively, which are in turn connected to the second and third branches 7 and 16, respectively, each tube being inserted into the corresponding tank 15 and 17 to draw the fluid from inside it on a control from the first control means 12.

Structurally, the second supply branch 7 may extend from the corresponding closure 18 to connect directly to the third branch 16 immediately downstream of the corresponding tube 21, so that the disinfectant/sterilizing fluids and the alternative fluid used instead of the first fluid can, thanks to corresponding second control means 22, be supplied alternately through a single second branch, which, in this case, is labeled 7 as a whole and is connected to the main line 1.

Looking in more detail, the second branch 7 (see FIG. 2 again) starts at the corresponding closure 18 and connects directly to the other closure 19 of the third branch 16 which supplies the alternative fluid.

At the top end of each of the tubes 20 and 21, the closures 18 and 19 are fitted with valve elements 23 and 24, respectively, designed to operate in conjunction with the second control means 22 to enable the corresponding fluid to flow, when required, into the single second branch 7 thus formed.

In particular, the closure 18 of the second branch 7 that supplies the disinfectant/sterilizing fluid has a channel 25 connected to the third branch 16 to convey the fluid in the direction of the other closure 19.

Similarly, the closure 19 of the third branch 16 that supplies the alternative fluid has a T-channel 26 (which in practice forms the third branch 16 itself) so as to allow the fluids to flow alternately into the second branch 7.

For operating safety and to guarantee that the two fluids do not mix, the closures 18 and 19 are preferably equipped with non-return valves 23 and 24, respectively, at the tops ends of the respective tubes 20 and 21.

The aforementioned second means 22 for controlling the alternate supply of the fluids comprise an air channel 29 made in each of the closures 18 and 19 operated by an appropriate unit 30 designed to generate pressure inside the respective tanks 15 and 17 so as to allow the supply of the selected fluid.

To further augment the safety of the water line, the second branch 7, in the section connecting the two closures 18 and 19, is equipped with second valve means 31 designed to safely shut off that section of the second branch 7 when the alternative fluid from the tank 17 is being used.

Purely by way of example, the alternative fluid may be purified water or purified water with patient-specific drugs added, or a sterile isotonic saline fluid.

The aforementioned first container 10 is used to hold the handpieces 5 or the ends of the conduits connecting with the dental unit (not illustrated since they are of customary type) during sterilization, disinfection or flushing of the line 1 or during cleaning of the entire water line. The first container 10 may form part of the dental unit (as shown in the drawing), being built into or mounted on the body of the dental unit, and is preferably equipped with a conduit 13 leading into a drain 14 which, in the embodiment illustrated and purely by way of example, is the drain of the dental unit, although separate drains may be provided, according to the specific requirements of the dental surgery.

Alternatively, the first container 10 may be a separate part 10′ applied to or positioned near the dental unit when treatments are being performed on the first branch 3.

The aforementioned switching means 11 act on the shutoff elements 6, 8 and 9 and are connected to the first control means 12.

The switching means 11 may be automatic or manual, operated by a manual action or activated automatically upon the occurrence of defined events on the dental unit and depending on the type of treatment to be performed. In other words, the switching means 11 interact with the first control means 12 to open and close the first, second and third shutoff elements 6, 8 and 9, through successive manual or automatic operating combinations and/or sequences that may be repeated two or more times, in such a way as to perform cycles of treatment on the first branch 3 with the first fluid 2 and/or alternatively with the second fluid conveyed by the second branch 7.

To allow these operations to be carried out, each of the first, second and third shutoff elements 6, 8 and 9 is connected to a respective operating unit 6v, 8v and 9v (of customary, pneumatic or electrical type) constituting the aforementioned switching means 11 connected to the first control means 12.

The first control means 12 may be of different types. If the dental unit is equipped with a microprocessor 32 to control its main and auxiliary functions, the first control means 12 may consist of a specific pushbutton 33 located on the dental unit (on a control console 32c), and controlled directly by the microprocessor 32 to allow the coordinated opening and closing of the shutoff elements 6, 8 and 9 according to preset parameters that can be stored in the microprocessor 32 and controlling the flow time and/or the quantity of the fluid.

Thanks to this possibility, the first control means 12 are activated by the microprocessor 32 according to defined parameters dependent on events performed on/by the dental unit (for example, resetting the position of the dentist's chair at the end of a treatment on a patient).

Alternatively, the control means 12 may consist of a pair of pushbuttons 34, 35 located on the dental unit together with other control pushbuttons (which are not illustrated). One of the pushbuttons 34, 35 is connected to settable timing means 36, illustrated as a block in the accompanying drawing. For example, without restricting the scope of the invention, the timing means 36 may consist of a timer or an air buffer with a settable valve at the discharge end to allow the air out in a preset time.

The timing means 36 are activated when the first shutoff elements 6 open or close or when the first branch 3 is full (that is, controlled directly by the dentist) and are designed to re-close or re-open the shutoff elements when the preset time has elapsed. The second pushbutton 35 may be connected to the second and third shutoff elements 8 and 9 to switch the supply of the main line 1 between the second branch 7 or the fluid source 2.

The apparatus described above offers the dentist a full range of functions for the treatment of patients. For example, instead of water from the mains, the patient may be supplied with a physiological saline solution or a medical fluid.

Further, the apparatus for selecting and changing the second fluid in the second branch allows sterilization/disinfection/flushing cycles and water line cleaning cycles to be carried out quickly and easily.

For example, the dentist can press the pushbutton 33 or 34 to act on the first shutoff element 6 according to the following single sequence of operations: opening for a preset time and/or quantity of first or third fluid until the first branch 3 is full of the selected fluid and the fluid is discharged from the handpieces 5, followed by the closing of the first shutoff elements 6 so as to perform a time flushing cycle on the first branch 3.

Another way of using the apparatus according to the invention is to switch fluid supply from the main line 1 to the second branch 7 so as to supply the first branch 3 with the second disinfectant/sterilizing fluid or with fluid used to flush the water line, place handpieces 5 in the first container 10, open the second shutoff element 8 and close the third shutoff element 9.

At this point, the first shutoff elements 6 can be opened for a preset time or quantity of the second fluid to completely renew the fluid in the water line, that is to say, in the first branch 3, until the fluid is discharged from the handpieces 5. The first shutoff elements 6 are then re-closed, again through the timing control or, depending on the treatment, by checking the quantity of the second fluid that is being used up.

After this cycle and, in some cases, after a preset time, during which the second fluid acts in the water line, the second and third elements 8 and 9 switch again, that is to say, the shutoff element 8 is closed and the first and third shutoff elements 9 are opened manually by pressing the pushbuttons 33 and 34 or automatically through a programmed sequence of the microprocessor 32 for a preset time and/or quantity of first or third fluid, so as to renew the fluid in the first branch 3, the first branch being flushed and the fluid being discharged from the handpieces 5. After flushing, the first shutoff elements 6 are re-closed to keep the first branch 3 in working conditions with a fluid used for medical treatment supplied through the second branch 7 by switching the control means 30 to turn off supply from the first container 15 and turn on supply of the third fluid from the second container 17.

As shown in the accompanying drawing, the first branch 3 may include, as one of the patient use-points 4, a fourth branch 37 that supplies a fluid to a tumbler 38 and that is equipped with a fourth shutoff element 39. Advantageously, the control means 12 can be connected to the fourth shutoff element 39 of the fourth branch 37, equipped with a switching unit 39v similar to the other switching units so that the fourth branch can be treated in the same way as the other branches.

An apparatus made according to the invention as described above thus achieves the aforementioned aims by providing a water line that is extremely simple, compact and economical in structure while at the same time offering a full set of functions for the treatment of patients and of the water line itself so as to keep the dental unit hygienically safe at a very high level of sterility.

Indeed, the possibility of choosing the alternative fluid to be used instead of water optimizes the use of the fluids to be used on the patient, while the possibility of supplying a fluid for disinfecting/sterilizing treatments of the water line itself, combined with the possibility of performing quick flushing cycles, rapidly restores the dental unit to hygienic working conditions.

The invention described can be subject to numerous modifications and variations without thereby departing from the scope of the inventive concept. Moreover, all the details of the invention may be substituted by technically equivalent elements.

What is claimed:

1. An apparatus for supplying and sanitizing the water line of a dental unit, such water line being of the type comprising:
a main fluid supply line connected at one end to at least one source of a first fluid and at the other end to the dental unit through at least one first branch for supplying a set of use-points comprising a plurality of handpieces;
first shutoff elements each located on the first branch that supplies the handpieces and each designed to turn on the supply of a handpiece when required;
the apparatus comprising:
at least one second branch connected and leading into the first branch supplying the use-points and conveying a second disinfectant/sterilizing fluid into the main line, the second branch and the main line being equipped with respective second and third shutoff elements designed to switch the supply of the first branch between the first fluid or the second disinfectant/sterilizing fluid coming from the second branch;
a first container for holding the handpieces or the ends of the conduits connecting with the dental unit during sterilization, disinfection, flushing or cleaning of the water line; and
switching means connected to control means that can be operated manually or triggered automatically upon the occurrence of defined events on the dental unit and, depending on the type of treatment, designed to determine successive manual or automatic operating combinations and/or sequences that may be repeated two or more times, during which the first, second and third shutoff elements are opened and closed in such a way as to perform cycles of treatment on the first branch with the first fluid and/or alternatively with the second fluid conveyed by the second branch.

2. The apparatus according to claim 1, wherein the switching means act on each of the first shutoff elements according to the following single sequence of operations:
opening for a preset time and/or quantity of first or second fluid until the first branch is full of the selected fluid and the fluid is discharged from the handpieces; and
closing of the first shutoff elements.

3. The apparatus according to claim 1, wherein the switching means act on each of the first, second and third shutoff elements in such a way as to perform the following operations in succession:
switching the supply of the first branch from the main line to the second branch through a combination or succession of operations to close the third shutoff element on the main line and to open the second shutoff element on the second branch;
opening the first shutoff elements for a preset time or quantity of fluid, so as to create a preset flow of the second fluid from the second branch into the first branch, and thus completely renewing the fluid in the first branch with the flow of the second fluid from the second branch until the second fluid is discharged from the handpieces;
closing the first shutoff elements after a preset time or when a preset quantity of fluid is finished;
further switching the supply of the first branch from the second branch to the main line through a combination or succession of operations to close the second shutoff element on the second branch and to open the third shutoff element on the main line so as to return to the condition in which the first branch is supplied with the first fluid;
opening the first shutoff elements for a preset time or quantity of fluid, so as to create a preset flow of the first fluid into the first branch, and thus completely flushing the first branch with the first fluid until the first fluid is discharged from the handpieces;
closing the first shutoff elements after a preset time or when a preset quantity of fluid is finished.

4. The apparatus according to claim 1, wherein the first container for the handpieces forms part of the dental unit, being built into or mounted on the body of the dental unit, and is equipped with a conduit leading into a drain.

5. The apparatus according to claim 1, wherein the first container for the handpieces is a separate part applied to or positioned near the dental unit when treatments are being performed on the first fluid supply branch.

6. The apparatus according to claim 1, wherein the second branch is supplied by a first tank which is independent and/or interchangeable.

7. The apparatus according to claim 1, further comprising a third branch that supplies an alternative third fluid, used instead of the first fluid, in the main line and in the first connecting branch, the third fluid being contained in a corresponding second tank.

8. The apparatus according to claims 1 and 7, wherein the second and third branches are equipped with respective closures used to connect up the first and second tanks of the fluids to be supplied and with respective tubes connected to the second and third branches, each tube being inserted into the corresponding tank to draw the fluid from inside it on a control from the first control means.

9. The apparatus according to claim 8, wherein the second supply branch starts at the corresponding closure and connects directly to the third branch closure immediately downstream of the corresponding tube, so that the disinfectant/sterilizing fluids and the alternative fluid used instead of the first fluid can, thanks to the second control means, be supplied alternately through a single second branch, which is connected to the main line.

10. The apparatus according to claim 9, wherein the second branch (7) extends from the corresponding closure and connects directly to the closure of the third branch that supplies the alternative fluid.

11. The apparatus according to claim 8 or 9, wherein the closures are fitted, at the top end of each of the tubes, with respective valve elements designed to operate in conjunction with the second control means to enable the corresponding fluid to flow, when required, into the single second branch.

12. The apparatus according to claim 10 or 11, wherein the closure of the second branch that supplies the disinfectant/sterilizing fluid has a channel connected to the third branch to convey the fluid in the direction of the other closure.

13. The apparatus according to claim 9, wherein the closure of the third branch that supplies the alternative fluid has a T-channel to allow the fluids to flow alternately into the main line.

14. The apparatus according to claim 11, wherein the closures are equipped with respective non-return valves, at the top ends of the respective tubes.

15. The apparatus according to claim 1, wherein the second means for controlling the supply of each of the fluids comprise an air channel made in each of the closures operated by an appropriate unit designed to generate pressure inside the respective tanks so as to allow the supply of the selected fluid.

16. The apparatus according to claim 8, wherein the second branch connecting the two closures is equipped with second valve means designed to safely shut off the second branch when the alternative fluid is being used.

17. The apparatus according to claim 7, wherein the alternative fluid is purified water.

18. The apparatus according to claim 7, wherein the alternative fluid is purified water with patient-specific drugs added.

19. The apparatus according to claim 7, wherein the alternative fluid is a sterile, isotonic, saline fluid.

20. The apparatus according to claims 6 and 7, wherein the first and second tanks are of the disposable type.

21. The apparatus according to claims 6 and 7, wherein the first and second tanks are of the type that can be sterilized and reused.

22. The apparatus according to claim 1, where the dental unit is equipped with a microprocessor to control its main and auxiliary functions, wherein the control means, consisting of a pushbutton located on the dental unit, are controlled by the microprocessor in such a way as to allow the coordinated opening and closing of the first, second and third shutoff elements according to preset parameters that can be stored in the microprocessor and that control the flow time and/or the quantity of the fluid.

23. The apparatus according to claim 22, wherein the first control means are activated by the microprocessor according to defined parameters dependent on events performed on/by the dental unit.

24. The apparatus according to claim 1, wherein the control means consist of a pair of pushbuttons located on the dental unit, one of which is connected to settable timing means that are activated when the first shutoff elements open or close or when the main line is full and that are designed to re-close or re-open the shutoff elements when the preset time has elapsed; the other pushbutton being connected to the second and third shutoff elements and being designed to switch from one to the other according to the treatments to be performed.

25. The apparatus according to claim 1, wherein each of the first, second and third shutoff elements is connected to an operating unit constituting the switching means connected to the control means.

26. The apparatus according to claim 1, where the first fluid supply branch comprises, as one of the patient use-points, a fourth branch that supplies a fluid to a tumbler and that is equipped with a fourth shutoff element, wherein the control means are also connected to the fourth shutoff element of the fourth branch, which is equipped with a switching unit so that the fourth branch can be treated in the same way as the other branches.
